(19) United States
(12) Patent Application Publication
(10) Pub. No.: US 2008/0281181 A1
(43) Pub. Date: Nov. 13, 2008

(54) Combination of Multi-Modality Imaging Technologies

(75) Inventors: James V. Manzione, Setauket, NY (US); Jerome Liang, Stony Brook, NY (US)

Correspondence Address: SCULLY SCOTT MURPHY & PRESSER, PC, 400 GARDEN CITY PLAZA, SUITE 300, GARDEN CITY, NY, 11530, US

(73) Assignee: THE RESEARCH FOUNDATION OF STATE UNIVERSITY OF NEW YORK, ALBANY (NY)

(21) Appl. No.: 11/587,467

(22) PCT Filed: May 13, 2005
(86) PCT No.: PCT/US05/16984

§ 371 Date: Mar. 24, 2008

Related U.S. Application Data
(60) Provisional application No. 60/571,366, filed on May 14, 2004.

(51) Int. Cl. A61B 5/05; A61B 6/00

(52) U.S. Cl. 600/407; 382/128

(57) ABSTRACT

Different applications for multiple imaging technologies are provided to advance patient care. In one aspect, a 3-D imaging device is used to identify the location of target region in a patient, such as a tumor or aneurysm, for instance. The location information is then used to control a device used in performing a surgical or other intervention, further imaging or a diagnostic or therapeutic procedure (920). The device can operate fully automatically, as a robot, or can assist a manual procedure performed by a physician. In another aspect, the invention provides a technique for obtaining an improved image of the vasculature in a patient (1020). In another aspect, images from multiple imaging technologies are combined or fused (1120) to achieve synergistic benefits.

### BACKGROUND OF THE INVENTION

1. Field of Invention

The invention relates generally to applications for multi-modality medical imaging technologies.

2. Description of Related Art

Medical imaging technologies include various types of 3-D and 2-D imaging systems. Systems using 3-D technologies include computed tomography (CT), magnetic resonance imaging (MRI), nuclear imaging, e.g., positron emission tomography (PET), and ultrasound, while systems using 2-D technologies include angiography, fluoroscopy, CT, MRI, nuclear imaging, and ultrasound. Each type of imaging technology has advantages and disadvantages with regard to tissue visualization and characterization, and factors such as patient comfort, health risks to the patient, and so forth.

Generally, the different imaging technologies have been used independently for narrowly tailored tasks due in part to the high costs of deploying and operating the imaging systems and limitations of the imaging systems. However, because of the increasing capabilities of imaging systems, more hospitals are expected to acquire and deploy multiple imaging technologies. Moreover, installation of the imaging devices in proximity to one another provides new opportunities to leverage the different imaging technologies to advance patient care.

### BRIEF SUMMARY OF THE INVENTION

The present invention addresses the above and other issues by providing different applications for multiple imaging technologies to advance patient care.

In one aspect, a 3-D imaging device is used to identify the location of a target region in a patient, such as a tumor or aneurysm, for instance. The location information is then used to control a device used in performing a surgical or other intervention, further imaging, or a diagnostic or therapeutic procedure. The device can operate fully automatically, as a robot, or can assist a manual procedure performed by a physician.

In another aspect, the invention provides a technique for obtaining an improved image of the vasculature in a patient, such as for optimizing visualization of an aneurysm.

In another aspect, images from multiple imaging technologies are combined or fused to achieve synergistic benefits.

The invention can be embodied in corresponding apparatuses, methods, program storage devices, and computer program products.

### BRIEF DESCRIPTION OF THE DRAWINGS

These and other features, benefits and advantages of the present invention will become apparent by reference to the following text and figures, with like reference numbers referring to like structures across the views, wherein:

FIG. 1a illustrates a top view of an installation using multiple imaging technologies.

FIG. 1b illustrates a side view of the CT installation of FIG. 1a.

FIG. 1c illustrates a side view of the angiographic imaging device of FIG. 1a.

FIGS. 2a-d are digital subtraction angiographic (DSA) images of an aneurysm examined at four different angles. The final image, FIG. 2d, demonstrates the neck of the aneurysm.

FIGS. 3a-e show an magnetic resonance angiogram (MRA) of the same patient as in FIG. 2. The smaller frames, FIGS. 3b and 3c, include only the image data for the involved vessel and the aneurysm. The data is freely rotated until the neck is properly visualized as shown in the final frame, FIG. 3e. Similar data sets can be acquired using a computed tomography angiogram (CTA).

FIGS. 4a-4l show images from twelve DSA studies that were required to obtain proper aneurysm neck visualization for this patient with conventional approaches.

FIGS. 5a-c show magnified views at various orientations of the patient treated in FIG. 4. The complex nature of the vasculature and difficulty in obtaining proper alignment is apparent.

FIGS. 6a-c show, respectively, images from a DSA of an aneurysm to be treated, unsubtracted aneurysm treated with coil placement, and final DSA post treatment showing exclusion of the aneurysm from circulation. FIG. 6a was selected from the series of 12 DSA projections from FIG. 4 that optimally demonstrates the relationship of the neck of the aneurysm with parent vessels.

FIGS. 7a-f show an outline of the formation of CTA for aneurysm characterization. FIG. 7a shows source CT data is acquired, FIG. 7b shows source data is compiled into a 3-D data set and maximum intensity projection (MIP) applied, FIG. 7c shows data is manually segmented to the area of interest. FIG. 7d shows additional manual bone segmentation, FIG. 7e shows the vascular model displayed with an anatomical (bone, in this case) reference, and FIG. 7f shows the vascular model displayed without the anatomical reference.

FIGS. 8a-d show a CTA showing the aneurysm and demonstrating the ability to change view orientation for optimized visualization of the aneurysm neck.

FIG. 9 shows a method for accessing a target region in a patient.

FIG. 10 shows a method for obtaining an improved image of vasculature in a patient.

FIG. 11 shows a method for fusing multiple images.

### DETAILED DESCRIPTION OF THE INVENTION

### Introduction

Innovations in technology have resulted in significant advances in angiography, computer tomography, magnetic resonance imaging, ultrasound, PET and nuclear imaging. The premise for combining various imaging modalities is that the fused technology will result in significant advancement in imaging horizons that would not otherwise be achievable.

Current Technologies include CT and standard single slice scanners. New advances include 2, 4, 16 and 64 slice scanners. For MRI, low, mid and high field scanners (0.5-3.0 Tesla) are available. For angiography, single and biplane angiographic equipment is available. The combined technologies will function in combination as a single unit when coupled together. When uncoupled, each unit will function separately and independently. In one possible example, a single or biplane angiographic suite is combined with a multi-slice CT scanner. In another possibility, a single or biplane angiography unit is combined with a 1.5 or 3.0 Tesla magnetic resonance scanner. It is also possible to combine angiography, CT, PET, nuclear and MRI scanners.

General applications include:

(a) Catheter directed 3-D-CTA and/or MRA.

(b) Digital subtraction CTA/3D-CTA.

(c) Organ/tissue blood flow, profusion and viability studies will now be able to be performed on the combined angiographic and multi slice CT, MRI, PET and/OR nuclear units.

(d) Tumor blood flow and perfusion.

(e) Blood brain barrier evaluation and manipulation studies.

(f) Image fusion of CT, MRI, PET, nuclear images and/or angiographic/fluoroscopic images for diagnosis, treatment planning and to enhance image guided procedures and development of robotic technology.

Installation of Multiple Imaging Systems in a Controlled Relationship to One Another

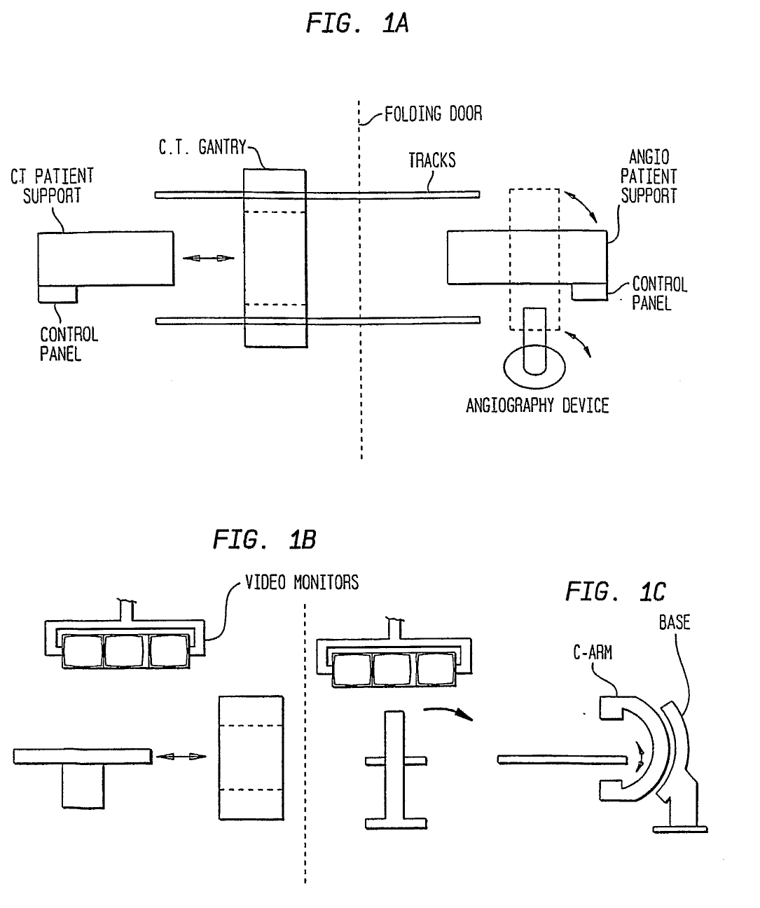

FIGS. 1a-c illustrates an installation using multiple imaging technologies. The setup is shown having two imaging technologies—CT and angiography. However, the concept can be extended to any type and number of imaging technologies, including, e.g., CT, MRI, PET, ultrasound, angiography and fluoroscopy. Moreover, other systems such as robots for performing procedures or interventions on patients can be incorporated into the installation. Various practical considerations should be accounted for when multiple imaging technologies are proximately located. For example, most metals cannot be used in a room having an MRI system.

The CT system can reside in its own scan room adjacent to the angiographic suite and can be used for routine clinical applications. When the system is required for neuro endovascular planning, for instance, the lead lined gymnasium wall of the scan room will open and the CT gantry will roll on the track system to encompass the digital subtraction angiographic (DSA) patient support. The patient will have already been catheterized. A three-dimensional CTA will be acquired via arterial or venous injection of contrast agent. After acquisition, the CT gantry will be wheeled back into its scan room and the lead wall again closed.

Thus, the CT gantry is moveable on tracks between a first room in which CT is performed, and a second room in which angiography is performed. Appropriate guide tracks, motors, sensors and actuators can be used for this purpose. A folding, lead lined door separates the two rooms to provide x-ray shielding to allow the two imaging systems to be operated at the same time. Moreover, the CT gantry can be quickly repositioned to be used sequentially with two patients that have been prepared and secured ahead of time on the CT patient support and the angio patient support, respectively. It is also possible for the patient supports to be on tracks to move relative to the CT gantry. However, having the gantry move to the patient avoids the need to move associated support equipment of the patient, such as intravenous drips and the like. Patient comfort is also optimized by minimizing movement of the patient.

Generally, the CT system can be used to diagnose and treat a wide variety of ailments, including head trauma, cancer and osteoporosis. A CT scan is used to form a three-dimensional image of a region of a patient. In a common design, an X-ray tube moves on a ring around an aperture through which the patient is positioned. An array of X-ray detectors is provided on the ring directly opposite the X-ray tube to detect the X-ray emissions. A motor turns the ring so that the X-ray tube and the X-ray detectors revolve around the body, thereby scanning from hundreds of different angles. Or, the X-ray tube can remain stationary while the X-ray beam is bounced off a revolving reflector. Each full revolution scans a narrow, horizontal slice of the body. Moreover, recently developed multi-slice CT scanners can scan multiple slices of the patient at the same time. The Siemens Somatom Sensation 16 CT scanner, for instance, is a 16-slice scanner. 64-slice scanners are also known. After the scanning, a computer combines data from each scan to form a detailed 3-D image of the scanned region of the patient's body that can be viewed on a workstation from different perspectives.

While CT scans provide accurate 3-D data of a patient at a point in time, angiographic imaging devices can be used to provide real-time continuous images, or a series of still images, while a physician performs an interventional procedure on the patient. For example, an angiographic imaging device can be used to perform a cerebral or coronary angiogram. A cerebral angiogram can be used to produce an image of the arteries in the brain or head to determine if the arteries are blocked by plaque or if a cerebral aneurysm is present. A contrast medium or dye is injected into specific arteries of the head or brain for which an image is desired. To achieve this, the physician inserts a catheter through a blood vessel such as the femoral artery, and feeds it to the desired artery. Once the catheter is in place, the contrast medium is injected and it mixes with the blood in the desired artery, thereby allowing the flow of the blood in the desired artery and other associated arteries to be imaged. The angiographic imaging device assists the physician by providing real-time feedback regarding the position and orientation of the catheter. However, as discussed in detail further below, for many procedures, it is necessary to take multiple image acquisitions using the angiographic imaging device to precisely visualize a condition. The angle and/or skew of the C-shaped arm of the device is repositioned for each acquisition under the control of an appropriate control system, actuators and the like.

In the installation of FIG. 1, the angio patient support can be rotated by ninety degrees so that a CT can be performed when the angio patient support is aligned with CT gantry, and an angiography can be performed when the angio patient support is aligned with the angiographic system. The angiographic imaging device typically includes an adjustable C-shaped arm with an x-ray tube and image intensifier on one side, and an x-ray receiver on the other side. The C-arm is adjusted to any specified position around the patient to obtain a 2-D image of the patient in a plane between the x-ray tube and receiver. Single plane or biplane imaging can be used. A fluoroscope can also be provided.

Appropriate control and display equipment can be provided to display information regarding the imaging systems. For example, a bank of video monitors can be provided in each imaging room. CT images can be viewed after the CT data is processed, which can take several seconds of processing. Angiographic images are available in real time, and are therefore useful in guiding the physician during a procedure such as catheter insertion. Workstations can be provided to display imaging data. Other computer and communications equipment for storing, processing and communicating data can be provided as will be apparent to those skilled in the art.

A control interface or panel (FIG. 1a) can be provided on or near the patient supports for use by the physicians in controlling movement of the supports, display of data on the monitors, and configuration of the respective imaging devices. The interface can include buttons, keys, a pointing device such as a mouse, a touch screen, voice control system, or the like.

The multiple imaging systems are arranged in a controlled relationship to one another and the patient. Advantageously, such as setup allows many new and improved imaging and other applications due to the speed with which the patient can be imaged by the different systems and the improved registration or alignment of the patient to each imaging system or other device such as a robot.

For instance, images from multiple imaging technologies can be combined or fused to achieve synergistic benefits. FIG. 11 shows a method for fusing multiple images. At block 1100, different imaging devices are arranged in a controlled relationship to one another to maintain an alignment of the target region with respect to each of the imaging devices. At block 1110, a target region of a patient is imaged using the different imaging devices. At block 1120, the different images are fused to obtain a fused or combined image.

In another example, a patient can be imaged with greater speed and accuracy since there is no need to setup the patient on a new support for each imaging system. Instead, the different imaging systems can be quickly moved to the patient, or the patient can be moved to the imaging systems. Moreover, health risks to the patient are reduced. A single injection remains in the patient's system long enough to permit imaging by the multiple imaging system. Furthermore, the patient can be repeatedly imaged by one or more given systems while in the middle of a procedure. Also, information can be exchanged between the different imaging systems, or between an imaging system and another system such as a robot, to assist the physician in performing a procedure. For example, a first imaging system can be used to detect the location of a target region of a patient, such as a tumor or aneurysm, and the location information can be used to automatically adjust the position of a second imaging system or robot to access the targeted region. A path to the target region can be determined using the location information, either by computer or manually, to access the target region while avoiding obstacles or non-targeted regions, such as nerves, the bowel and blood vessels. The path essentially provides directions to assist the robot or physician in accessing the target region while avoiding the obstacles. The path determined can be the shortest safe path.

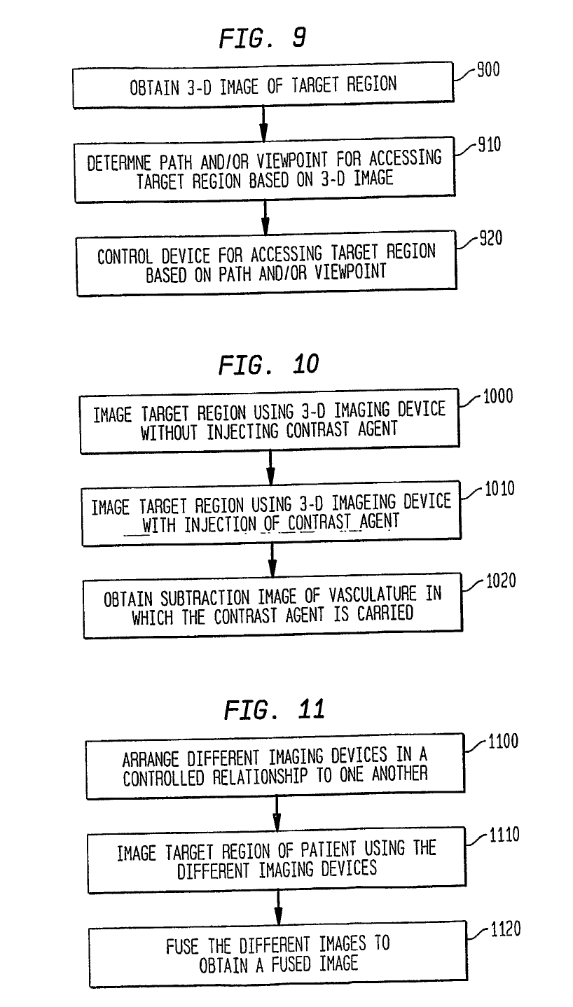

The above process is summarized in FIG. 9, which shows a method for accessing a target region in a patient. At block 900, one or more 3-D images of a target region are obtained. At block 910, a path and/or viewpoint for accessing the target region is determined based on the one or more 3-D images. At block 920, a device for accessing the target region is controlled based on the path and/or viewpoint.

Moreover, a 2D or 3D virtual image of a target can be generated to permit accessing the target by some route such as a transvascular or percutaneous route. For example, a CTA can be used to provide a blood vessel map that is superimposed on a fluoroscope screen. The physician can view the progress of a catheter, for instance, on the screen as it moves toward a target. The virtual image of the blood vessel is used as a map to the target region of the patient.

In one possible example that uses the installation of FIG. 1, a stroke victim that is admitted to a hospital can be imaged on the CT system to determine if there is hemorrhaging in the brain. If there is none, the angiographic station can be used to image the patient while performing a revascularization. In turn, the CT system can be used to measure cerebral blood flow prior to the procedure to determine its appropriateness and during the revascularization to guide the revascularization procedure. The series of activities can be carried out very quickly since the patient remains in the same location and the different imaging devices can be used within minutes of one another.

Using CT/MRI System to Obtain Data for Reducing the Number of Angiograms that are Needed to Visualize an Aneurysm

Advantageously, by interfacing imaging systems, data from one system can be used to facilitate the use of a second system. This can result in profound improvements in patient care. The following discussion illustrates the point by showing how a CT system can be used to obtain data for reducing the number of angiograms that are needed to visualize an aneurysm, thereby reducing risk to the patient and improving patient care.

Endovascular embolization therapy has become an alternative to surgery for the treatment of intracranial aneurysms. While the efficacy of endovascular coil embolization is clear¹⁻³, the procedure is based on old technology and could be optimized based on new technology to enhance efficiency, while reducing risk and cost.

A fundamental aspect of both endovascular embolization and surgical clipping is the requirement for a series of selective digital subtraction angiographic (DSA) studies to determine: aneurysm location (parent and efferent arteries), aneurysm neck size, the relationship of the neck of the aneurysm to the parent vessel and the morphology of the aneurysm sac. Proper aneurysm neck visualization allows for the placement of embolizing materials such as coils within the aneurysm and not within the lumen of the involved vessel. Vessel embolization would produce catastrophic consequences (stroke). It also allows for surgical planning in patients who undergo surgical aneurysm clipping. Associated with these multiple DSA studies are high doses of iodinated contrast, significant radiation exposure, extended periods with catheters in the neurovasculature and long procedure times.

The invention reduces or avoids the above-mentioned disadvantages by removing the requirement of repeated DSA studies thereby improving the efficiency and efficacy of this procedure. In one possible approach, a sixteen-slice CT system is coupled to the angiographic DSA hardware to allow for the production of a high quality three-dimensional computed tomography angiography (CTA). This high quality CTA allows for full evaluation of the neurovascular and automatic positioning of the x-ray tube/image intensifier assembly of the angiographic (DSA) unit for proper visualization of the aneurysm neck. Proof of this hypothesis will improve the efficacy and efficiency of endovascular embolization therapy and optimize preoperative planning.

To prove the hypothesis, a high quality CTA such as a sixteen slice CT system should be used for the rapid production of thin slices, the application of arterially delivered contrast agent and high photon acquisition (increased kV and mAs). Software algorithms can be developed that allow for automatic positioning of the x-ray tube/image intensifier assembly based on the three-dimensional CTA generated from the CT to optimize the aneurysm neck visualization. Furthermore, the efficiency and efficacy of this new configuration can be evaluated for planning endovascular embolization therapy and surgical aneurysm clipping.

Egas Moniz performed the first intra-arterial use of iodinated contrast agent to roentgenographically visualize the vessels of the brain in 1927⁴. Since this time, evaluation of the neurovasculature has matured to include digital subtraction angiography (DSA)⁵. In this method, iodinated contrast agent is delivered as a bolus through an arterial catheter. Digital images from a conventional image intensifier are collected before the arrival of contrast (mask) and at contrast arrival. Mask image data is subtracted pixel by pixel from the image data containing iodine. The result is an image of the iodine-containing vasculature.

Using DSA, a wide variety of vascular conditions and diseases can be diagnosed and assessed, and therapies can be directed.

A cerebral aneurysm is a ballooning of a weakened region of a blood vessel. If left untreated, the aneurysm can continue to weaken until it ruptures and bleeds into the head. In 1991, Guglielmi first described the technique of occluding aneurysms using detachable coils placed by endovascular approach. Packing the aneurysm with these coils, excludes it from the circulation. Given the catastrophic potential of subarachnoid hemorrhage, this new endovascular therapy, has become an alternative to surgical clipping.

Each year approximately 15,000 patients are treated in the United States for aneurysmal subarachnoid hemorrhage¹⁰. Of this group, it is estimated that 20 percent are treated by endovascular coil embolization rather than traditional surgical approaches¹⁰. As advances in vascular imaging and embolization technology are made, endovascular embolization therapy is likely to increase the portion of aneurysms treated. Recent innovations in this treatment have included: embolizing coils that allow complex 3D shapes¹¹, combinational use of coils and stents¹², the use of balloons¹³ and aneurysm filling polymer agents¹⁴.

Initial selective DSA studies are used to determine: aneurysm location (parent and efferent arteries), aneurysm neck size and morphology of the sac. A critical aspect of endovascular embolization, however, is visualization of the neck of the aneurysm during treatment. This ensures that the coils occlude the aneurysm and not the parent vessel. To accomplish this, the x-ray tube/image intensifier of the angiographic imaging system assembly must be properly positioned. Conventionally, proper positioning is achieved using several DSA acquisitions. After each acquisition, the angle and skew of the assembly is adjusted and through an educated trial-and-error process, proper positioning is eventually achieved.

Unfortunately, significant iodinated contrast can be required in the initial selective DSA studies and subsequent x-ray tube/image intensifier alignment DSA studies so there are concerns regarding nephrotoxicity¹⁵. Another concern associated with DSA is the risk of brain injury¹⁶. Each selective DSA acquisition requires the placement of a catheter in a different artery and each could result in stroke or vascular damage. Radiation exposure is another important risk of this procedure. Long fluoroscopy times and many DSA frames are often required for these combined diagnostic and therapeutic procedures. Cases of skin reactions ranging from mild erythema to skin necrosis have been documented in the literature¹⁷⁻²². Moreover, the procedure is costly since it requires a special technical team including an invasive radiologist, an anesthesiologist, nursing support and technologist.

Attempts are ongoing to solve the aneurysm neck visualization problem. One of the most advanced attempts employs three-dimensional DSA²³⁻²⁴. This technique requires contrast injection during a 180-200 degree rotational acquisition. The rotation of the x-ray tube/image intensifier assembly requires 5-10 seconds. From an initial mask, reconstruction of a 3D vascular model can be accomplished and used for proper alignment for aneurysm neck visualization. Unfortunately, the mask and contrast data sets are separated by the order of minutes. As a result, the mask and iodine data may not be aligned resulting in a vascular model that is not useful. In addition, three-dimensional DSA requires four selective arterial catheterizations to fully examine neurovascular anatomy and to determine the location of the aneurysm. This conventional approach therefore exposes the patient to significant risks. The ideal imaging technology for neurovascular aneurysms will employ a minimum number of selective studies to reduce the chance of stroke¹⁶ and nephrotoxicity¹⁵.

Computed tomography (CT) allows for the visualization of thin axial slices of anatomy. Through the use of intravenous iodinated contrast agents, vasculature can be separated from soft tissue using maximum-intensity projection (MIP) analysis²⁵. High-density tissues, such as bone are usually manually segmented. Through volume rendering, a stack of two-dimensional data can be displayed as a three-dimensional computed tomography angiogram (CTA). The image quality is improved using newer spiral CT because acquisition is faster so the contrast levels are higher, a volume of data is rapidly acquired allowing thinner slices for improved CTA resolution, and thin slices also minimize partial volume artifacts. Employing this technology, CTA can visualize 2-3 mm aneurysms with sensitivity of 77-97% and specificity of 87-100%²⁶.

Moreover, helical CTA with intraarterial contrast administration (ICA) is superior to three-dimensional DSA in the evaluation of the aneurysmal neck. Three-dimensional DSA clearly defined the neck in slightly more than half the aneurysms studied while helical CTA with ICA showed the aneurysm neck in all cases²⁷. Helical CTA with ICA was also superior to three-dimensional DSA in defining arterial branches adjacent to the aneurysm²⁷.

Multi-slice CT will further revolutionize aneurysm evaluation by virtue of the availability of thinner slices and faster acquisitions²⁸. The improved spatial resolution enables for high quality 3D visualization and reveals equivalent morphologic information when compared to invasive angiography²⁸. With the proliferation of this technology, DSA use will probably become limited to arterial catheter placement for the purpose of treatment.

Still there are a variety of challenges to overcome in using multi-slice CTA for planning endovascular coil embolization or surgical repair. For example, the contrast afforded by intravenous iodine delivery is inconsistent. The resulting CTA may, therefore, not be of sufficient quality to plan embolization therapy. Arterial delivery of contrast agent and the rapid acquisition of thin slices will provide much improved CTA image quality. Another key issue is visualization of vascular pathologies such as aneurysms at the skull base. Often CT data in this region contains streak artifacts. One source of these artifacts is photon starvation of projection data through bony areas²⁹⁻³⁰. The use of high technique can reduce this artifact²⁹ as well as the application of improved reconstruction methods for discontinuous projection data³⁰. Other sources of these artifacts include partial volume averaging³¹ and beam hardening³². Thin slices have proven useful in reducing streak artifacts by minimizing partial volume averaging³¹. Methods that compensate for beam hardening are also under investigation³².

Since CTA provides a three-dimensional view of the neurovasculature, the diagnostic CTA often helps with initial x-ray tube/image intensifier positioning. Unfortunately, conventionally, several DSA acquisitions are often still required for proper intensifier positioning and to provide improved visualization of the vascular anatomy. In principle, a CTA with appropriate feducial markers could be taken in the CT suite and then the patient moved to special procedures for DSA. Unfortunately, the quality of intravenous CTA is not sufficient for full characterization of the neurovasculature and it may be difficult to properly realign the feducial markers. An essential feature that will allow CTA to replace DSA for planning is arterially delivered contrast for improved vascular contrast and a coupled CT and DSA system that provides automatic registration between CTA and DSA studies, e.g., as shown in FIG. 1.

Installation of Multiple Imaging Systems Allow Contrast Agent to be Delivered in Aorta Rather than in Arteries in Head or Brain

According to the invention, as discussed above, coupling a sixteen-slice CT system optimized for CTA to DSA hardware is expected to eliminate multiple DSA planning studies. A further advantage is that, in this arrangement, a catheter could be placed within the aorta rather than the arteries in the head or brain using the conventional angiographic hardware. Generally, a contrast agent can be delivered in the aorta or other proximal blood vessel rather than selectively in arteries/veins in a target region prior to imaging the target region. This results in significant advantages, including a reduced risk to the patient. Once placed, the contrast agent could be delivered arterially, allowing for the production of a high quality CTA. This improved image quality could be obtained due to a combination of: higher arterial concentration of iodine, the use of very thin slices (<1 mm), high kilovoltage and tube current providing sufficient photon statistics and improved image processing. The resulting high quality CTA could be used to examine the neurovasculature in detail and to automatically position the x-ray tube/image intensifier assembly of the angiographic hardware for proper aneurysm neck visualization because patient geometry is unchanged. Using such a coupled system, the efficacy and efficiency for planning endovascular embolization therapy or surgery could be greatly improved.

Automatic Positioning of an Imaging System Based on Data Obtained from Another Imaging System.

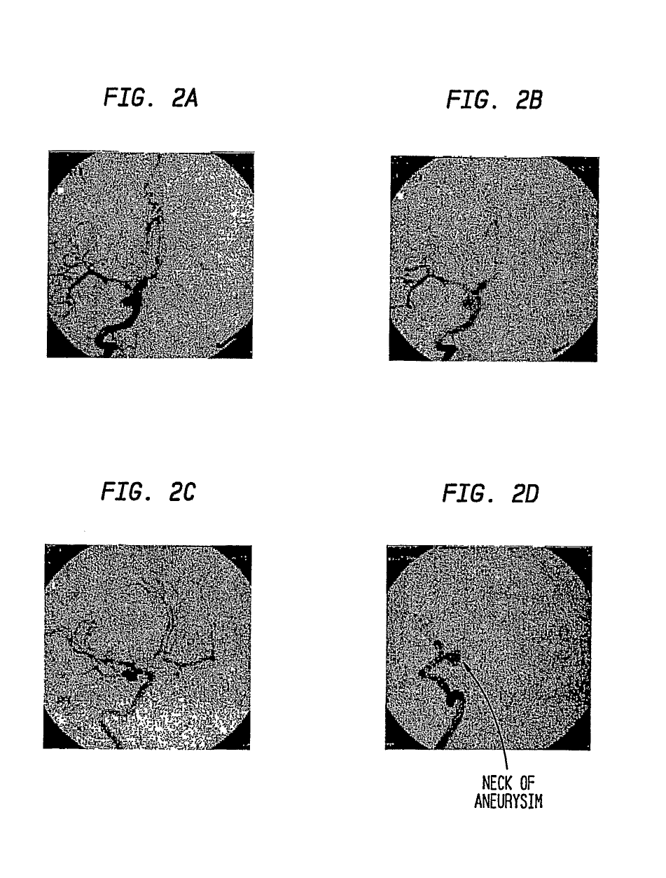

Preliminary studies indicate that the key limiting factors of efficiency and efficacy of endovascular embolizations are: the volume of iodinated contrast used, patient radiation exposure, the number of selective catheterizations required, the time the patient is anesthetized and total procedure time. All these factors are significantly controlled by the ability to position the x-ray tube/image intensifier assembly to properly visualize the neck or bulge of the aneurysm. This problem is demonstrated in FIGS. 2a-d. Four representative DSA projections are shown, with the aneurysm visible in each. Unfortunately, embolization therapy can only be safely applied if the aneurysm, neck and parent vessel are seen separately with no overlap, as shown in FIG. 2d. Without this view, successful embolization is not possible because: a) the neck diameter cannot be properly assessed so the appropriate coil(s) may not be chosen, b) the embolization materials could be inadvertently placed in the parent artery rather than the aneurysm, resulting in stroke, and c) it will be impossible to tell when the aneurysm sac is filled.

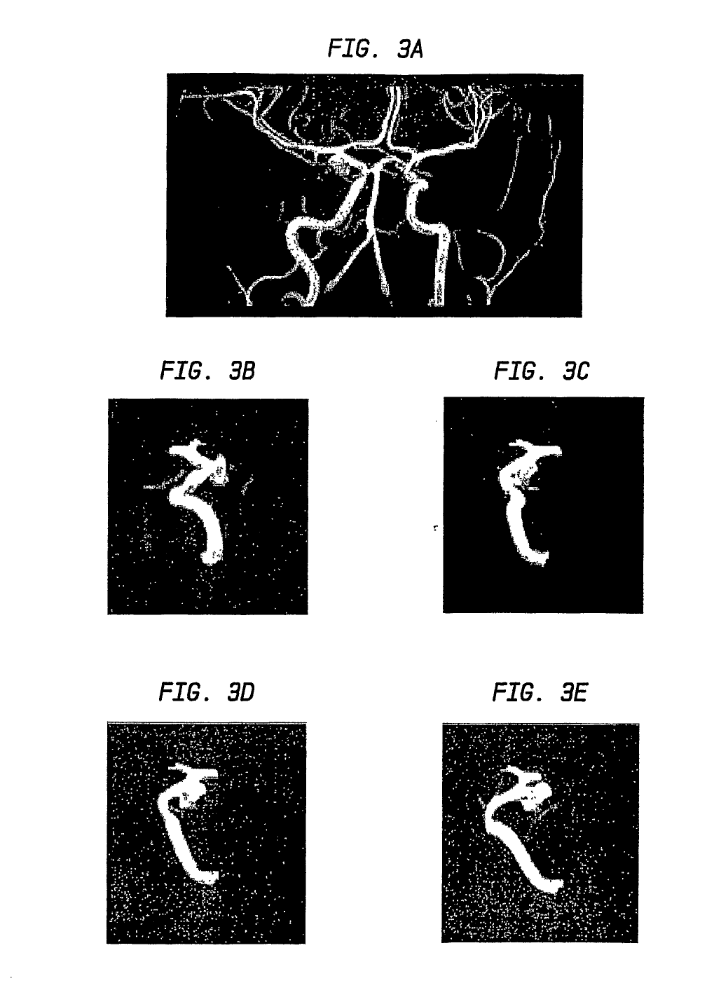

In principle, the proper orientation for aneurysm neck visualization can be directly determined from any three dimensional angiographic technique (e.g., CTA or MRA). FIGS. 3a-e show the diagnostic magnetic resonance angiogram (MRA) acquired for the same patient as in FIG. 2.

The aneurysm is clearly seen. Interrogating the image data of the involved vessel from a variety of angles allows for the selection of proper angulation for aneurysm neck visualization, the final small frame of FIG. 3e. We propose to extract from the three dimensional angiographic CT data the proper orientation for neck visualization, and automatically apply that angulation and skew to the x-ray tube/image intensifier assembly. In practice, the physician can select the proper orientation manually by viewing the 3-D images from the CT scan, and selecting a viewpoint that provides a desired orientation using any type of user interface. The 3-D image rendering software associated with the CT scanner can store orientation or positional data that is associated with the desired orientation or orientations. The positional data can then be used by the 2-D angiographic device by operating a motor to automatically position the device accordingly. For user convenience, multiple orientations that are designated by the physician while viewing the 3-D images can be associated with push buttons on a control associated with the 2-D device to allow the physician to manually command the 2-D device to move to the corresponding positions.

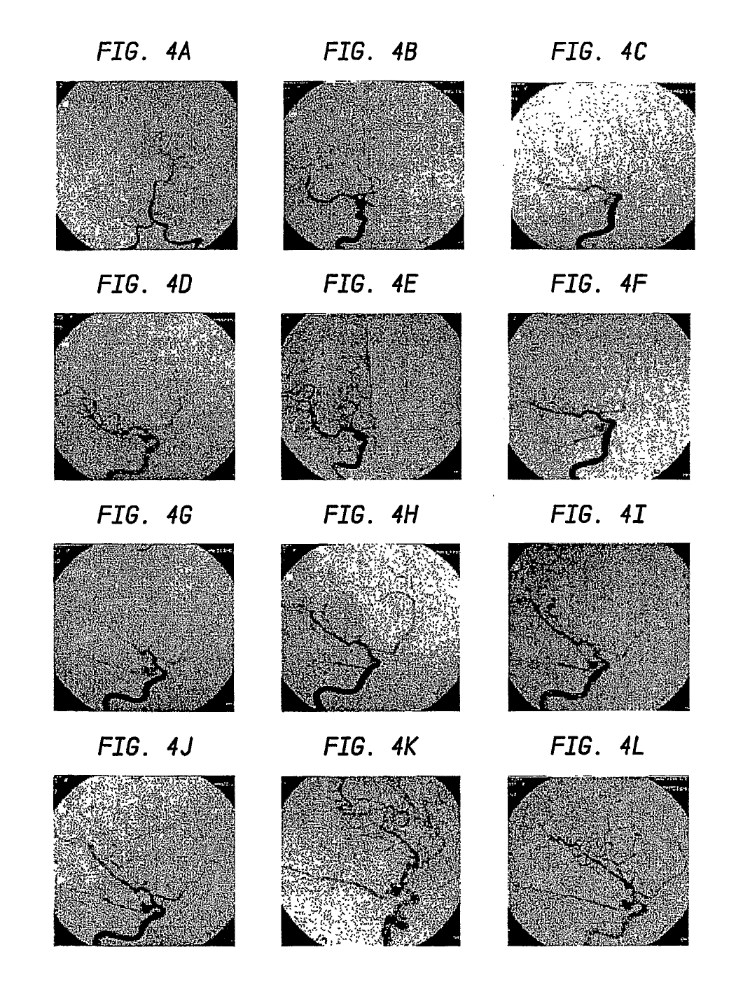

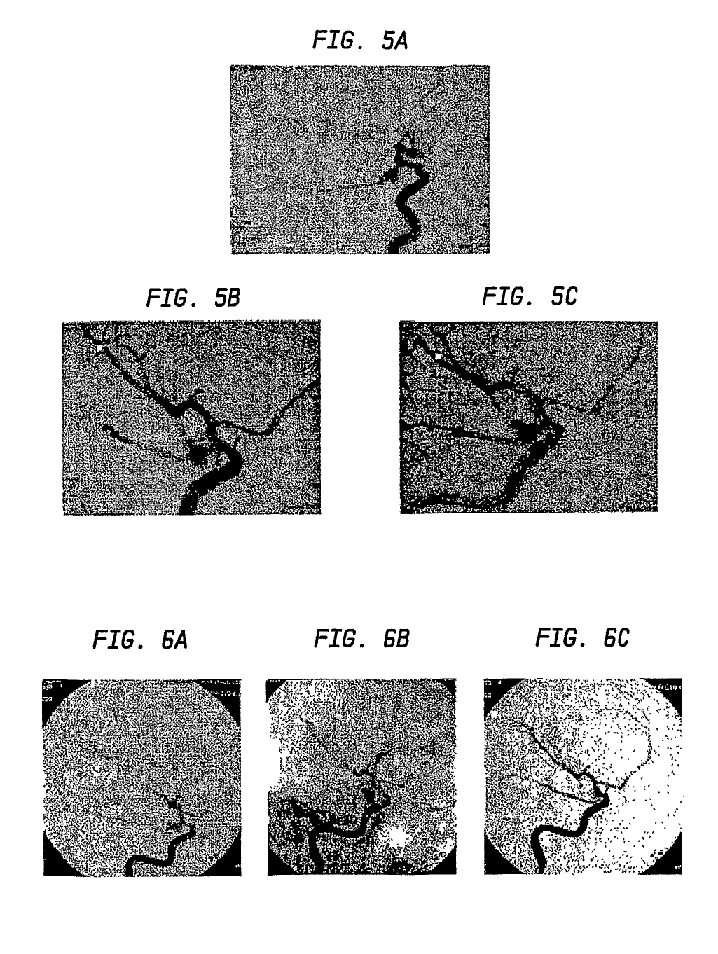

Having the capability for automatic positioning according to the invention is therefore a significant advantage. For example, in many instances, biologic variant and aneurysm location makes proper manual x-ray tube/image intensifier assembly positioning very difficult. FIGS. 4-6 demonstrate the successful treatment of such a case. This patient presented with fetal origin of the posterior communicator artery from the right internal carotid artery. As a result of aneurysm location and this biological variation, planning was very difficult and, as can be seen, for this patient, twelve DSA acquisitions were required to properly align the x-ray tube/image intensifier for aneurysm neck visualization.

FIG. 6a was selected by the physician from the series of 12 DSA projections from FIG. 4 that optimally demonstrate the relationship of the neck of the aneurysm with parent vessels. Notice the very close proximity of the posterior cerebral artery (arrow) to the neck of the aneurysm. This degree of delineation of the aneurysm from surrounding vessels is essential to avoid accidental closure of normal vessels. Using FIG. 6a as the reference DSA projection during the endovascular coiling procedure permitted safe closure of the aneurysm with preservation of the normal surrounding cerebral vessels. FIG. 6b is the non-subtracted post endovascular treatment result. FIG. 6c is the subtracted counterpart of FIG. 6b; the aneurysm is closed and the posterior cerebral artery is preserved (arrow).

While both MRA and CTA can provide the appropriate data for complete neurovascular characterization and x-ray tube/image intensifier alignment, coupling an MRA system to a DSA system would be a more difficult engineering task because of the associated magnetic fringe field. Coupling a sixteen-slice or other multi-slice CT system to a DSA system is by comparison a simpler task and has been chosen for this application.

Referring back to FIG. 1, the sixteen-slice CT scanner can reside in its own scan room, adjacent to the special procedures DSA suite, and can be used for routine clinical applications. When the CT system is required for endovascular embolization treatment planning, a lead lined gymnasium wall separating these two rooms will open and the gantry will roll on tracks to encompass the DSA patient support. The patient will have already been catheterized under fluoroscopic guidance of the DSA system. A three-dimensional CTA will be acquired via arterial injection of iodinated contrast agent. After acquisition, the CT gantry will be wheeled back into its scan room and the lead wall again closed.

The integration of multi-slice CT and DSA can be a very cost effective solution to the problem of aneurysm treatment planning. It should be noted that the price of a biplane rotational three-dimensional DSA unit approaches that of the integrated system proposed here with the added advantage that combined unit can be used in an integrated fashion as well as separately and independently.

A Subtraction Technique for Obtaining an Improved Image of the Vasculature in a Patient Using a Multi-Slice CT

Computed Tomographic Angiography (CTA) has become a noninvasive method of evaluating blood vessels of the body. It is often utilized to assess the intracranial circulation (blood vessels of the brain). However, a limiting factor in utilizing this technique in evaluating the intracranial circulation is the bone structures at the skull base. In these regions the bone has to be manually removed from the image. This is both time consuming and subject to human error. As a result, reliable images of the blood vessels as they pass through the skull base to the brain are not consistently or reproducibly obtainable using current technology. Digital subtraction techniques have been utilized to remove bone from digitally acquired angiographic images obtained on conventional angiographic equipment. Using a similar concept, subtracted CT images demonstrating the blood vessels at the skull base should be obtainable since CT images are digitally acquired.

This technique will resolve a significant limitation of current technology. The new technique will provide a reliable and consistent automated method of visualizing blood vessels of the brain as they pass through the skull base. This technique should significantly enhance current technology, which utilizes inconsistent and unreliable user dependent methods to visualize blood vessels in these areas.

A non-contrast scan of the skull base is needed in addition to the CTA contrast study. This will result in small increase in radiation dose compared to conventional CTA technique. However the improved visualization of the blood vessels with this new technique should preclude the need for more invasive studies (e.g., conventional angiography) and their associated risks.

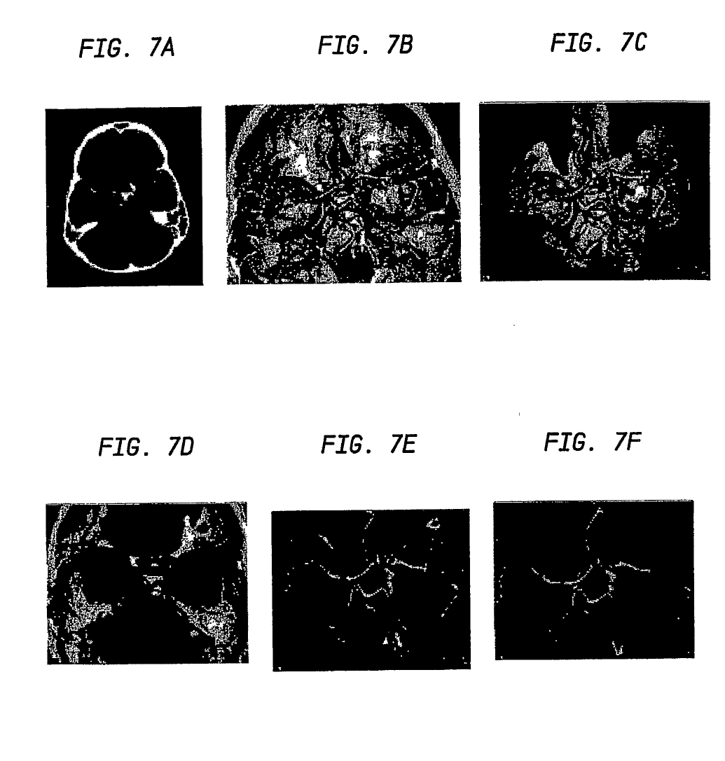

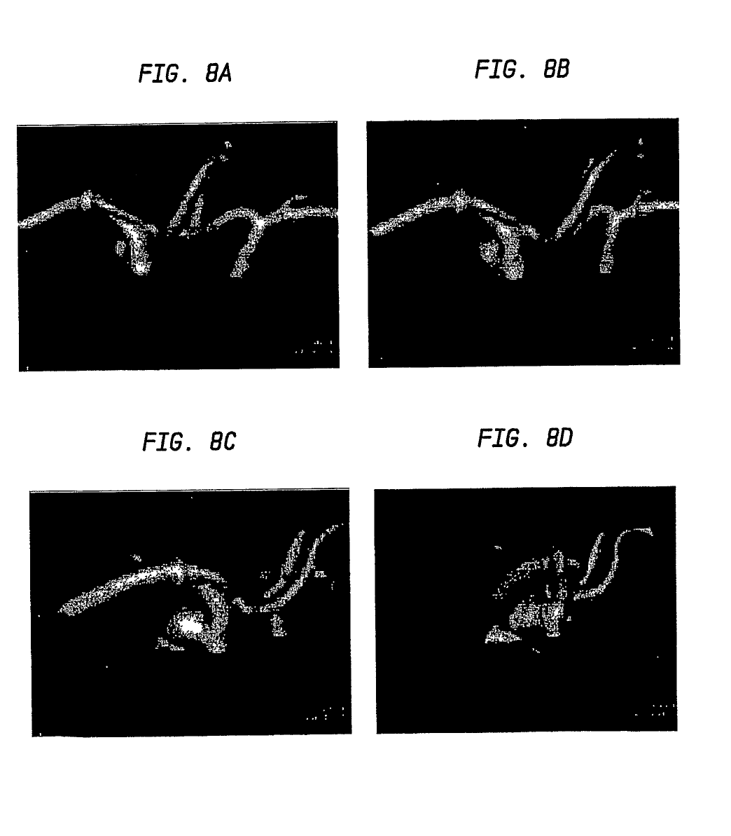

In a specific example, while it has been our experience that multi-slice CTA is excellent for visualization and study of the neck of the aneurysm, some technical challenges must be overcome. These challenges are illuminated in the process steps of a clinical CTA that documents the location of an aneurysm as shown in FIGS. 7 and 8. The steps taken in forming the CTA are outlined in FIG. 7. Initially, source CT images are acquired following intravenous iodinated contrast administration. FIG. 7a shows the location of the aneurysm in the source data. This data is then compiled as a three-dimensional data set and soft tissue is removed by applying maximum intensity projection (MIP). Following MIP, the opacified vasculature and bone remain (FIG. 7b). The bone is next removed manually by a skilled operator (FIGS. 7c-d) leaving only the vasculature (FIG. 7e), which can then be displayed with an anatomical reference (FIG. 7e). FIG. 8 shows how the final CTA data can be manipulated to provide the geometry that optimizes aneurysm neck visualization.

With the image quality demonstrated above for multi-slice CTA, its use for guidance of neuroembolization has significant potential over conventional DSA from many different points of view: fewer selective studies, potentially less iodinated contrast, potentially less radiation exposure, potentially shorter period under anesthesia and a shorter procedure time. Pitfalls in the application of multi-slice CTA include the requirement for manual segmentation of bone. Clearly, errors in this task can have significant clinical impact. Accordingly, a further aspect of the invention involves using subtraction techniques to remove the requirement of manual segmentation.

In particular, digital subtraction angiography (DSA) improves the visualization of contrast containing vasculature by subtracting overlying structures. Analogous CTA techniques would eliminate the need for manual segmentation but have not been developed because of misregistration between the mask and the iodinated contrast-containing image. This misregistration is due to the required movement of the patient through the gantry for the mask and the inability to precisely match these positions during the contrast acquisition. For a small region (skull base) using a sixteen-slice or multi-slice CT system, this limitation is removed. CT images can be collected from the small volume without moving the patient just before and just after arterial administration of iodinated contrast agent in an analogous fashion as is used in DSA. Temporal resolution the order of two seconds should be attainable.

Such subtracted images will have all soft tissue and bone eliminated providing only the signal from iodine in an analogous fashion to DSA. The advantage is that MIP can be applied without the difficulty of manual segmentation of bone.

A key issue is contrast-to-noise ratio following such CT subtraction. Fortunately, arterial contrast delivery will ensure adequate contrast. Therefore, excessive radiation exposure is not required for noise reduction in this application.

The initial proof of concept of the potential for subtraction CT can be undertaken on phantoms. Specifically, the RMI head CT phantom (Model-Gammex 461A) can be employed. This phantom is composed of water equivalent plastic and a superficial bone equivalent ring. Inserts in this phantom allow the placement of tubes containing solutions. We will acquire stationary three-dimensional (sixteen slice) phantom studies with blood equivalent solutions (Houndsfield number=30) and with dilute solutions of iodine (Houndsfield number=40, 50, 100, 200, 300). The iodine containing image data will be reconstructed and subtracted from the phantom image data containing the blood equivalent solution. Maximum intensity projection will be applied and the resulting image examined. For successful subtraction, the MIP image should only include the tube of iodine.

The above process can be summarized as indicated in FIG. 10, which shows a method for obtaining an improved image of vasculature in a patient. At block 1000, a target region is imaged using a 3-D imaging device without injecting a contrast agent. At block 1010, the target region is imaged using the 3-D imaging device with injections of a contrast agent. At block 1020, a subtraction image of the vasculature in which the contrast agent is carried is obtained based on differences between the images obtained with and without the contrast agent.

Software Tools

CTA of the entire brain will be acquired as previously describe: arterial injection of contrast agent and rapid acquisition of the whole brain using a sixteen-slice CT. In cases where the aneurysm is near the skull base, a modified CTA will also be acquired of the skull base. This acquisition will include high technique and subtraction techniques as previously described. In order to provide this information coherently, both data sets will be combined into a composite three-dimensional model. To this end, software tools can be developed, which allow replacing the skull base region of the brain CTA with the skull base CTA.

Automatic Positioning and Control of a Robot Based on Data Obtained from an Imaging System

As discussed, when multiple imaging systems are integrated, imaging data from a first imaging system can be used to control a second imaging system and/or other device such as a robot. The robot can have one or more arms that are used for various purposes, including performing a procedure on a patient either automatically or by assisting a physician. For example, the robot can advance a needle or a biopsy device into the patient, e.g., to obtain a biopsy of a liver tumor. The robot arm can also employ a surgical tool or endoscope, for instance.

Furthermore, the robot can adapt to movement of the patient's chest caused by breathing. While the head can be fixed relatively immobile, respiration of the patient results in significant movement of the chest, abdomen and internal organs. The robot can track this movement by imaging and tracking three markers on the patient, such as on the patient's chest, and adjust its movements accordingly. The CT or other 3-D imaging device can also obtain data regarding the movement of the patient due to respiration. A respiratory cycle can be observed along with the associated movements of the patient. For example, data averaged over five cycles can be used. The robot can then use the data obtained by the CT or other imaging system to compensate its movements and anticipate the patient's movements.

### LITERATURE CITED

1. Johnston S C. Higashida, R T. Barrow D L. Caplan, L R. Dion, J E. Hademenos G. Hopkins L N. Molyneux, A. Rosenwasser, R H. Vinuela F. Wilson C B. Recommendations for the Endovascular Treatment of Intracranial Aneurysms: A Statement for Healthcare Professionals from the Committee on Cerebrovascular Imaging of the American Heart Association Council on Cardiovascular Radiology, Stoke, Volume 33(10) 2002. p 2536-2544.
2. Bederson J B. Awad I A. Wiebers D O. Piepgras D. Haley E C Jr. Brott T. Hademenos G. Chyatte D. Rosenwasser R. Caroselli C. Recommendations for the management of patients with unruptured intracranial aneurysms: A statement for healthcare professionals from the Stroke Council of the American Heart Association. [Guideline. Journal Article. Practice Guideline] Circulation. 102(18):2300-8, 2000.
3. Johnston S C. Zhao S. Dudley R A. Berman M F. Gress D R. Treatment of unruptured cerebral aneurysms in California. Stroke. 32(3):597-605, 2001.
4. Mistretta C A. Grist T M. X-ray digital subtraction angiography to magnetic resonance-digital subtraction angiography using three-dimensional TRICKS. Historical perspective and computer simulations: a review. [Review] [43 refs] [Historical Article. Journal Article. Review. Review, Tutorial] Investigative Radiology. 33(9):496-505, 1998.
5. Ducos De Lahitte M, Manelfe C, Marc Vernes J P, Rascol A, Bonafe' A, Guiraud B., Intravenous film subtraction angiography. In: Mistretta C, Crummy A B, Strother C M, Sackett J F. eds. Digital subtraction arteriaography: An application of computerized fluoroscopy. Chicago, Ill.: Yearbook Medical Publishers, 1981.
6. Guglielmi G, Vinuela F, Sepetka I, et al. Electrothrombosis of saccular aneurysms via endovascular approach, part 1: electrochemical basis, technique, and experimental results. J Neurosurg. 1991; 75: 1-7.
7. Schievink W I. Intracranial aneurysms. N Engl J Med. 1997; 336: 28-40.
8. Graves E J. Detailed diagnoses and procedures, National Hospital Discharge Survey, 1990. Vital Health Stat 13. 1992; 113: 1-225.
9. Hop J W, Rinkel G J, Algra A, van Gijn J. Case-fatality rates and functional outcome after subarachnoid hemorrhage: a systematic review. Stroke. 1997; 28: 660-664
10. Hopkins L N, Lanzino G, Guterman L R. Treating complex nervous system vascular disorders through a “needle stick”: origins, evolution, and future of neuroendovascular therapy. Neurosurgery. 2001; 48: 463-475.
11. Malek A M, Higashida R T, Phatouros C C, Dowd C F, Halbach V V. Treatment of an intracranial aneurysm using a new three-dimensional-shape Guglielmi detachable coil: technical case report. Neurosurgery. 1999; 44: 1142-1144.
12. Malek A M, Halbach V V, Phatouros C C, Lempert T E, Meyers P M, Dowd C F, Higashida R T. Balloon-assist technique for endovascular coil embolization of geometrically difficult intracranial aneurysms. Neurosurgery. 2000; 46: 1397-1406.
13. Sekhon L H, Morgan M K, Sorby W, Grinnell V. Combined endovascular stent implantation and endosaccular coil placement for the treatment of a wide-necked vertebral artery aneurysm: technical case report. Neurosurgery. 1998; 43: 380-383.
14. Yang X, Wu Z, Li Y, Tang J, Sun Y, Liu Z, Yin K. Re-evaluation of cellulose acetate polymer: angiographic findings and histological studies. Surg Neurol. 2001; 55: 116-122.
15. Waugh J R, Sacharias N. Arteriographic complications in the DSA era. Radiology 1992; 182: 243-246.
16. Leffers A M. Wagner A. Neurologic complications of cerebral angiography. A retrospective study of complication rate and patient risk factors. ACTA Radiologica. 41(3):204-10, 2000.
17. D'incan M. Roger H. Gabriliargues J. Mansard S. Parent S. Chazal J. Irthum B. Souteyrand P. [Radiation-induced temporary hair loss after endovascular embolization of the cerebral arteries: six cases]. [French] [Journal Article] Annales de Dermatologie et de Venereologie. 129(5 Pt 1): 703-6, 2002 May.
18. Rad effects Food and Drug Administration. Public Health Advisory: Avoidance of Serious X-Ray-Induced Skin Injuries to Patients During Fluoroscopically-Guided Procedures. Sep. 30, 1994. Rockville, Md., Center for Devices and Radiological Health, FDA, 1994.
19. Huda W, Peters K R. Radiation-induced Temporary Epilation after a Neuroradiologically Guided Embolization Procedure. Radiology 1994: 193: 642-644.
20. Scheinman M M. NASPE Survey on Catheter Ablation. PACE 1995; 18: 1474-1478.
21. Wagner L K, Eifel P J, Geise R A. Potential Biological Effects Following High X-ray Dose Interventional Procedures. JVIR 1994; 5: 71-84.
22. Food and Drug Administration. Important Information for Physicians and Other Healthcare Professionals: Recording Information in the Patient's Medical Record That Identifies the Potential for Serious X-ray-Induced Skin Injuries Following Fluoroscopically Guided Procedures. Sep. 15, 1995. Rockville, Md., Center for Devices and Radiological Health, FDA, 1995.
23. Imakita S. Onishi Y. Hashimoto T. Motosugi S. Kuribayashi S. Takamiya M. Hashimoto N. Yamaguchi T. Sawada T. Subtraction CT angiography with controlled-orbit helical scanning for detection of intracranial aneurysms. AJNR: American Journal of Neuroradiology. 19(2):291-5, 1997.
24. Castano-Duque C H, Ruscalleda-Nadal J et al. Early experience studying cerebral aneurysms with rotational and three-dimensional angiography and review of CT and MR angiography literature, Interventional Neuroradiology 8: 377-391, 2002.
25. M I P Prokop M. Shin H O. Schanz A. Schaefer-Prokop C M. Use of maximum intensity projections in CT angiography: a basic review. Radiographics. 17(2):433-51, 1997.
26. Hope J K A, Wilson J L, Thomson F J. Three dimensional CT angiography in the detection and characterization of intracranial berry aneurysms. AJNR Am J Neuroradiol. 1996; 17: 439-445.
27. Nomura M, Kida S, et. al. Pre-embolization study of ruptured cerebral aneurysms with helical CT. Interventional Neuroradiology 5 (suppl 1): 219-223, 1999.
28. Wintersperger B J. Helmberger T K. Herzog P. Jakobs T F. Waggershauser T. Becker C R. Reiser M F. New abdominal CT angiography protocol on a 16 detector-row CT scanner—first results. Radiologe. 42(9):722-7, 2002.
29. Robertson D, Weiss P, Fishman E, Magid D, Walker P. Evaluation of CT techniques for reducing artifacts in the presence of metallic orthopedic implants. J Comput Assist Tomogr 1988; 121: 236-41.
30. Hsieh J. Adaptive streak artifact reduction in computed tomography resulting from excessive x-ray photon noise. Medical Physics. 25(11):2139-47, 1998.
31. Alberico R A. Loud P. Pollina J. Greco W. Patel M. Klufas R. Thick-section reformatting of thinly collimated helical CT for reduction of skull base-related artifacts. AJR. American Journal of Roentgenology. 175(5):1361-6, 2000.
32. Chen C Y. Chuang K S. Wu J. Lin H R. Li M J. Beam hardening correction for computed tomography images using a postreconstruction method and equivalent tissue concept. Journal of Digital Imaging. 14(2):54-61, 2001.
33. Radiochromic Film Dosimetry, AAPM Radiation Therapy Committee Task Group #55 (1998).
34. Kuan H M, Lawson W E, Manzione J V, and Ferretti J A, Birk J W and Dignam V A. Patient skin dose in interventional procedures by a portal film technique. RSNA 2000.
35. Performance Evaluation and Quality Assurance in Digital Subtraction Angiography Diagnostic X-Ray Imaging Committee/Digital Radiography/Fluorography AAPM Task Group (1985).
36. Phantoms for Performance Evaluation and Quality Assurance of CT Scanners. AAPM Monogram (1977).

The invention has been described herein with reference to particular exemplary embodiments. Certain alterations and modifications may be apparent to those skilled in the art, without departing from the scope of the invention. The exemplary embodiments are meant to be illustrative, not limiting of the scope of the invention.

What is claimed is:

1. A method for positioning a device for accessing a target region in a patient, comprising:
determining (910) at least one of a particular path and a particular viewpoint for accessing the target region based on at least one 3-D image of the target region obtaining by imaging the target region; and
controlling (920) the device for accessing the target region according to the at least one of a particular path and a particular viewpoint.

2. The method of claim 1, wherein:
the device for accessing the target region comprises a radiographic imaging system.

3. The method of claim 1, wherein:
the at least one 3-D image of the target region is obtained from a 3-D imaging system employing at least one of computed tomography (CT), magnetic resonance imaging (MRI), positron emission tomography (PET), and ultrasound.

4. The method of claim 1, wherein:
the at least one 3-D image of the target region comprises a fusion of different images from different 3-D imaging systems employing at least one of computed tomography (CT), magnetic resonance imaging (MRI), positron emission tomography (PET), and ultrasound.

5. The method of claim 1, wherein:
the at least one of a particular path and a particular viewpoint for accessing the target region is determined manually by a physician by viewing the at least one 3-D image.

6. The method of claim 1, wherein:
the at least one of a particular path and a particular viewpoint for accessing the target region is determined automatically using software that evaluates the at least one 3-D image.

7. The method of claim 1, wherein:
the device for accessing the target region comprises a robot.

8. The method of claim 1, wherein:
the device for accessing the target region is used in performing at least one of: (a) a surgical or other intervention, (b) further imaging, and (c) a diagnostic and/or therapeutic procedure; and
the device operates automatically, as a robot.

9. The method of claim 1, wherein:
the device for accessing the target region is used in performing at least one of: (a) a surgical or other intervention, (b) further imaging, and (c) a diagnostic and/or therapeutic procedure; and
the device assists a manual procedure performed by a physician.

10. The method of claim 1, further comprising:
arranging in a controlled relationship to one another, (a) an imaging device for imaging the target region, (b) the device for accessing the target region, and (c) a patient support.

11. The method of claim 1, further comprising:
imaging the target region using a CT system to obtain data for reducing the number of angiograms that are needed to visualize an aneurysm.

12. The method of claim 1, wherein the target region is in the head or brain of the patient, further comprising:
delivering a contrast agent in the aorta rather than in arteries in the head or brain prior to imaging the target region.

13. The method of claim 1, wherein the target region is anywhere in the body of the patient, further comprising:
delivering a contrast agent in the aorta or other proximal blood vessel rather than selectively in arteries and/or veins in the target region prior to imaging the target region.

14. The method of claim 1, further comprising:
generating at least one of a 2D and a 3D virtual image of the target region to permit accessing the target region by at least one of a transvascular route and a percutaneous route.

15. A program storage device tangibly embodying software instructions which, when executed by at least one processor, perform a method for positioning a device for accessing a target region in a patient, the method comprising:
determining (910) at least one of a particular path and a particular viewpoint for accessing the target region based on at least one 3-D image of the target region obtaining by imaging the target region; and
controlling (920) the device for accessing the target region according to the at least one of a particular path and a particular viewpoint.

16. A method for obtaining an image of vasculature in a target region in a patient, comprising:
imaging (1000) the target region using a 3-D imaging device without injection of a contrast agent to obtain a first image;
imaging (1010) the target region using the 3-D imaging device with injection of the contrast agent to obtain a second image; and
obtaining (1020) a subtraction image comprising the vasculature in which the contrast agent is carried based on differences between the first and second images.

17. The method of claim 16, wherein:
the 3-D imaging device comprises a multi-slice CT scanner.

18. The method of claim 16, wherein:
both the first and second images are obtained without repositioning the patient with respect to the 3-D imaging device.

19. A program storage device tangibly embodying software instructions which, when executed by at least one processor, perform a method for obtaining an image of vasculature in a target region in a patient, the method comprising:
accessing a first image obtained by imaging the target region using a 3-D imaging device without injection of a contrast agent (1000);
accessing a second image obtained by imaging the target region using the 3-D imaging device with injection of the contrast agent (1010); and
obtaining a subtraction image comprising the vasculature in which the contrast agent is carried based on differences between the first and second images (1020).

20. A method for providing an image of a target region of a patient, comprising:
imaging (1110) the target region of the patient using different imaging devices to obtain different images;
wherein the different imaging devices are arranged in a controlled relationship to one another (1100) to maintain an alignment of the target region with respect to each of the imaging devices; and
fusing (1120) the different images to obtain a fused image.

21. The method of claim 20, wherein:
the different images are 3-D images obtained from different 3-D imaging systems employing at least one of computed tomography (CT), magnetic resonance imaging (MRI), positron emission tomography (PET), and ultrasound.

22. A program storage device tangibly embodying software instructions which, when executed by at least one processor, perform a method for providing an image of a target region of a patient, the method comprising:
accessing different images obtained by imaging the target region of the patient using different imaging devices (1110);
wherein the different imaging devices are arranged in a controlled relationship to one another (1100) to maintain an alignment of the target region with respect to each of the imaging devices; and
fusing (1120) the different images to obtain a fused image.
